(19) United States
(12) Patent Application Publication
(10) Pub. No.: US 2003/0229261 A1
(43) Pub. Date: Dec. 11, 2003

(54) Cardiac support devices and methods of producing same

(75) Inventors: Michael J. Girard, Lino Lakes, MN (US); Kay Ann Nauertz, South St. Paul, MN (US); Jose F. Nunez, St. Anthony, MN (US); James Cox, Hamel, MN (US); Holly Hicks, West St. Paul, MN (US)

Correspondence Address: ACORN CARDIOVASCULAR, INC., P.O. BOX 2903, MINNEAPOLIS, MN, 55402, US

(73) Assignee: Acorn Cardiovascular, Inc.

(21) Appl. No.: 10/165,509

(22) Filed: Jun. 6, 2002

(51) Int. Cl. A61N 1/362

(52) U.S. Cl. 600/16

(57) ABSTRACT

A pattern for a three dimensional cardiac support device that includes a proximal edge having a concave shape; and a seam edge that includes: first and second sides, wherein said first and second sides are substantially symmetrical along a vertical axis, first and second upward inflection regions, first and second downward inflection regions; and an intersection point. A three dimensional cardiac support device that includes: a pattern, wherein said pattern is substantially symmetrical along a vertical axis, and wherein said pattern includes a proximal edge having a concave shape, and a seam edge that includes: first and second sides, first and second downward inflection regions, first and second downward inflection regions, and an intersection point; and a seam wherein the seam is formed by folding the pattern across the vertical axis and joining the seam edge to itself. A three dimensional cardiac support device formed from a two-dimensional material, wherein the three-dimensional cardiac support device has seams having a seam length, and a surface area, wherein the ratio of the seam length to the surface area is from about 0.1/inch to about 0.2/inch.

### I. BACKGROUND OF THE INVENTION

A. Field of the Invention

The invention relates to three-dimensional objects that are formed from two-dimensional materials and methods of producing the same. More specifically, the invention relates to a cardiac support device made from a two-dimensional sheet of material that has reduced material distortion and a lower ratio of seam distance to surface area of the support, and methods of producing them.

B. Description of the Prior Art

Congestive heart disease is a progressive and debilitating illness, characterized by a progressive enlargement of the heart. As the heart enlarges, it must perform an increasing amount of work in order to pump blood during each heartbeat. In time, the heart becomes so enlarged that it cannot adequately supply blood to the body. An afflicted patient is fatigued, unable to perform tasks requiring even a minimum level of exertion and experiences pain and discomfort. Further, as the heart enlarges, the internal heart valves may not adequately close. This impairs the function of the valves and further reduces the heart's ability to supply blood to the body.

Causes of congestive heart disease are not fully known. In some instances, congestive heart disease may result from certain viral infections. In such cases, the heart may enlarge to such an extent that even after the viral infection has passed the disease continues its progressively debilitating course. Congestive heart disease and treatment methodologies are described, for example in U.S. Pat. No. 6,123,662, issued Sep. 26, 2000 entitled CARDIAC DISEASE TREATMENT AND DEVICE, the disclosure of which is incorporated herein by reference.

Congestive heart failure has an enormous societal impact. In the United States alone, about five million people suffer from the disease. Alarmingly, congestive heart failure is one of the most rapidly accelerating diseases (about 400,000 new patients in the United States each year). Economic costs of the disease have been estimated at $38 billion annually.

One type of treatment can be found in commonly assigned U.S. Pat. No. 5,702,343 to Alferness dated Dec. 30, 1997. It teaches a jacket that constrains cardiac expansion during diastole. Also, PCT International Publication No. WO 98/29401 published Jul. 9, 1998 teaches a cardiac constraint in the form of surfaces on opposite sides of the heart with the surfaces joined together by a cable through the heart or by an external constraint. U.S. Pat. No. 5,800,528 dated Sep. 1, 1998 teaches a passive girdle to surround a heart. German utility model DE 295 17 393 describes a non-expansible heart pouch. PCT International Publication No. WO 98/58598 published Dec. 30, 1998 describes a cardiac pouch with an elastic limit.

A cardiac support device can be placed on an enlarged heart and fitted snug during diastole. For example, a knit jacket device can be loosely slipped on the heart. After such placement, the material of the jacket can be gathered to adjust the device to a desired tension. The gathered material can be sutured or otherwise fixed to maintain the tensioning. The heart may be pre-shrunk prior to placement of the device or the device may be fitted on the heart without pre-shrinking the heart. The device is adjusted for a snug fit on the heart during diastole.

### II. SUMMARY OF THE INVENTION

The invention provides, a pattern for a three dimensional cardiac support device is disclosed that includes a proximal edge having a concave shape; and a seam edge that includes: first and second sides, wherein said first and second sides are substantially symmetrical along a vertical axis, first and second upward inflection regions, first and second downward inflection regions; and an intersection point.

The invention also provides a three dimensional cardiac support device is disclosed that includes: a pattern, wherein said pattern is substantially symmetrical along a vertical axis, and wherein said pattern includes a proximal edge having a concave shape, and a seam edge that includes: first and second sides, first and second downward inflection regions, first and second downward inflection regions, and an intersection point; and a seam wherein the seam is formed by folding the pattern across the vertical axis and joining the seam edge to itself.

The invention further provides a method of making a three-dimensional cardiac support device from a two-dimensional material where the three-dimensional cardiac support device has a ratio of seam length to surface area from about 0.1/inch to about 0.2/inch.

The invention also provides a three-dimensional cardiac support device produced from a two-dimensional material wherein the three-dimensional cardiac support device has a ratio of seam length to surface area of about 0.10/inch to about 0.20/inch.

### III. BRIEF DESCRIPTION OF THE DRAWINGS

FIG. 1 is a schematic cross-sectional view of a normal, healthy human heart shown during systole;

FIG. 1A is the view of FIG. 1 showing the heart during diastole;

FIG. 2 is a schematic cross-sectional view of a diseased human heart shown during systole;

FIG. 2A is the view of FIG. 2 showing the heart during diastole;

FIG. 3 is a perspective view of one embodiment of a cardiac support device;

FIG. 3A is a side elevation view of a diseased heart in diastole with the support device of FIG. 3 in place;

FIG. 4 is a plan view of a pattern in accordance with one aspect of the invention;

FIG. 4a is a plan view of a pattern in accordance with one aspect of the invention illustrating measurement of a radius of curvature;

FIG. 5 is a plan view of another pattern in accordance with one aspect of the invention; and

FIG. 6 is a plan view of yet another pattern in accordance with one aspect of the invention.

### IV. DESCRIPTION OF THE PREFERRED EMBODIMENT

A. Congestive Heart Disease and the Heart

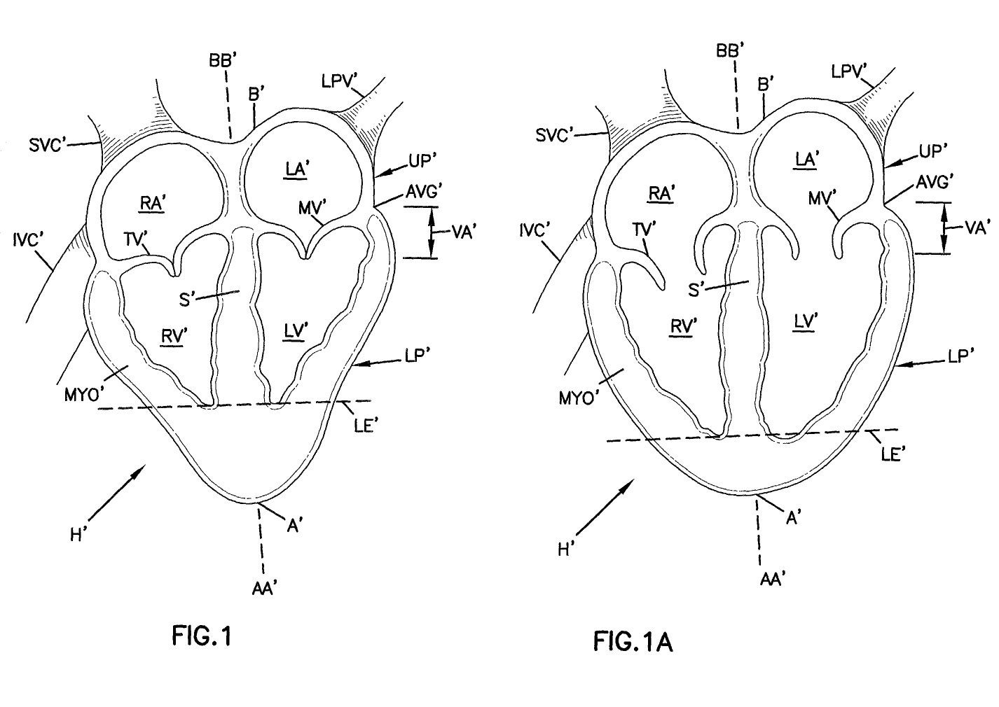

With initial reference to FIGS. 1 and 1A, a normal, healthy human heart H′ is schematically shown in cross-section and will now be described in order to facilitate an understanding of the present invention, and the complex shape of the heart H′. In FIG. 1, the heart H′ is shown during systole (i.e., high left ventricular pressure). In FIG. 1A, the heart H′ is shown during diastole (i.e., low left ventricular pressure).

The heart H′ is a muscle having an outer wall or myocardium MYO′ and an internal wall or septum S′. The myocardium MYO′ and septum S′ define four internal heart chambers including a right atrium RA′, a left atrium LA′, a right ventricle RV′ and a left ventricle LV′. The heart H′ has a length measured along a longitudinal axis BB′-AA′ from an upper end or base B′ to a lower end or apex A′.

The right and left atria RA′, LA′ reside in an upper portion UP′ of the heart H′ adjacent the base B′. The right and left ventricles RV′, LV′ reside in a lower portion LP′ of the heart H′ adjacent the apex A′. The ventricles RV′, LV′ terminate at ventricular lower extremities LE′ adjacent the apex A′ and spaced there from by the thickness of the myocardium MYO′.

Due to the compound curves of the upper and lower portions UP′, LP′, the upper and lower portions UP′, LP′ meet at a circumferential groove commonly referred to as the A-V (atrio-ventricular) groove AVG′. Extending away from the upper portion UP′ are a plurality of major blood vessels communicating with the chambers RA′, RV′, LA′, LV′. For ease of illustration, only the superior vena cava SVC′, inferior vena cava IVC′ and a left pulmonary vein LPV′ are shown as being representative.

FIGS. 1 and 1A show a normal, healthy heart H′ during systole and diastole, respectively. During systole (FIG. 1), the myocardium MYO′ is contracting and the heart H′ assumes a shape including a generally conical lower portion LP′. During diastole (FIG. 1A), the heart H′ is expanding and the conical shape of the lower portion LP′ bulges radially outwardly (relative to axis AA′-BB′).

The motion of the heart H′ and the variation in the shape of the heart H′ during contraction and expansion is complex. The amount of motion varies considerably throughout the heart H′. The motion includes a component that is parallel to the axis AA′-BB′ (conveniently referred to as longitudinal expansion or contraction). The motion also includes a component perpendicular to the axis AA′-BB′ (conveniently referred to as circumferential expansion or contraction).

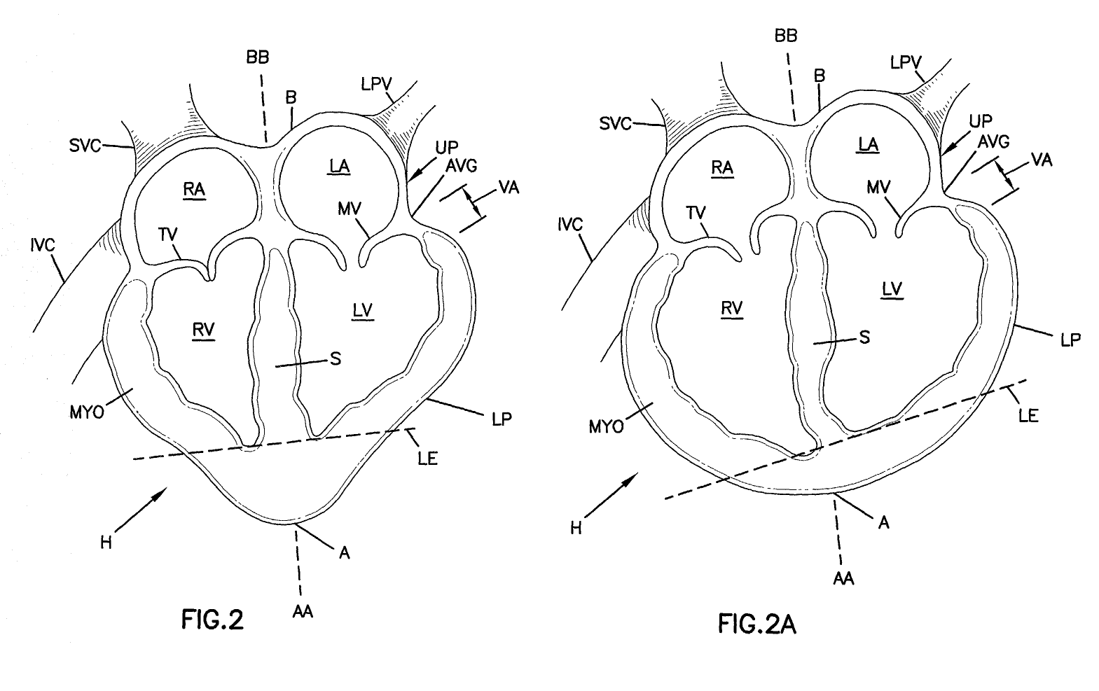

Having described a healthy heart H′ during, systole (FIG. 1) and diastole (FIG. 1A), comparison can now be made with a heart H deformed by congestive heart disease. Such a heart H is shown in systole in FIG. 2 and in diastole in FIG. 2A. All elements of diseased heart H are labeled identically with similar elements of healthy heart H′ except for the omission of the apostrophe in order to distinguish diseased heart H from healthy heart H′. Comparing FIGS. 1 and 2 (showing hearts H′ and H during systole), the lower portion LP of the diseased heart H has lost the tapered conical shape of the lower portion LP′ of the healthy heart H′. During diastole (FIG. 2A), the deformation of heart H is even more extreme.

B. Cardiac Support Therapy

Having described the heart, and the characteristics and problems of congestive heart disease, a treatment method and apparatus are described, such as that disclosed in U.S. Pat. No. 6,085,754, issued Jul. 11, 2000. In general, a cardiac support device, or a jacket is configured to surround the myocardium MYO. While the method of the present invention will be described with reference to a jacket as described in U.S. Pat. No. 6,085,754, it will be appreciated that the present invention is applicable to any cardiac support device including those shown in U.S. Pat. No. 5,800,528 and PCT International Publication No. WO 98/29401.

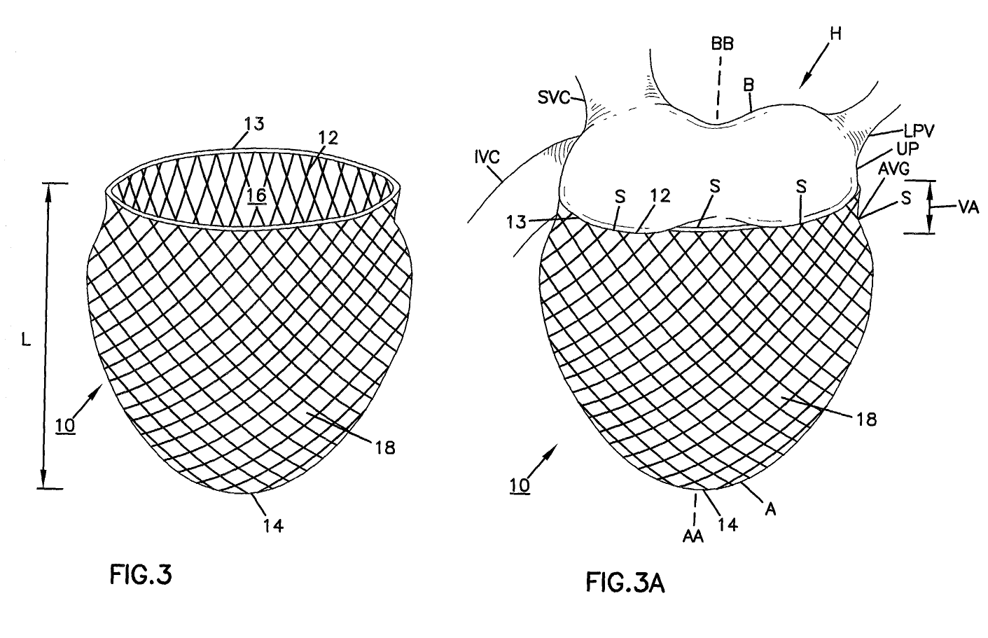

With reference now to FIGS. 3, and 3A, an example of a cardiac support device is shown as a jacket 10, 10′ of flexible, biologically compatible material. The jacket 10, 10′ is an enclosed material having upper and lower ends 12, 12′, 14 and 14′. Upper end 12, 12′ is further defined by upper rim 13. The jacket 10, 10′ defines an internal volume 16, 16′ which is completely enclosed but for the open ends 12, 12′ and 14′. In the embodiment of FIG. 3, lower end 14 is closed

The jacket 10 is dimensioned with respect to a heart H to be treated. Specifically, the jacket 10 is sized for the heart H to be constrained within the volume 16. The jacket 10 can be slipped around the heart H. The jacket 10 has a length L between the upper and lower ends, 12 and 14, sufficient for the jacket 10 to constrain the lower portion LP. The upper end 12 of the jacket 10 extends at least to the A-V groove AVG and further extends to the lower portion LP to constrain at least the lower ventricular extremities LE.

In one embodiment, the jacket 10 material comprises intertwined fibers, for example, fibers intertwined as a knit or weave. In one embodiment, the jacket 10 is constructed from a knit material. Preferably the jacket 10 is constructed from a compliant, biocompatible material. As used herein, the term “compliant” refers to a material that can expand in response to a force. “Compliance” refers to the displacement per a unit load for a material. “Elasticity” refers to the ability of the deformed material to return to its initial state after the deforming load is removed.

The fabric 18 is preferably tear and run resistant. In the event of a material defect or inadvertent tear, such a defect or tear is restricted from propagation by reason of the knit construction.

C. Cardiac Support Device Pattern

Having described important characteristics of the heart, diseases thereof, and a cardiac support device for treatment thereof, a method of constructing such a device and devices constructed thereby in accordance with the invention will now be discussed. Such a method is discussed in reference to a jacket such as that disclosed in U.S. Pat. No. 6,085,754, issued Jul. 11, 2000, however the devices and methods of the invention are not limited thereby.

It is generally desirable when constructing such a cardiac support device to reduce distortion of the material and reduce the number of seams present in the jacket. Reducing distortion of the material can improve functioning and continuity of the jacket from one area to another. Such improved functionality may be due to more constant forces being applied across the surface that the jacket is contacting. Reducing the number of seams in the jacket can reduce irritation and/or abrasion that may be caused by the jacket, thereby reducing fibrosis and scar tissue. Reducing the distortion and the seam length simultaneously can be difficult because the starting material (i.e. fabric) is a two-dimensional material and the final product (i.e. jacket) is a three-dimensional article.

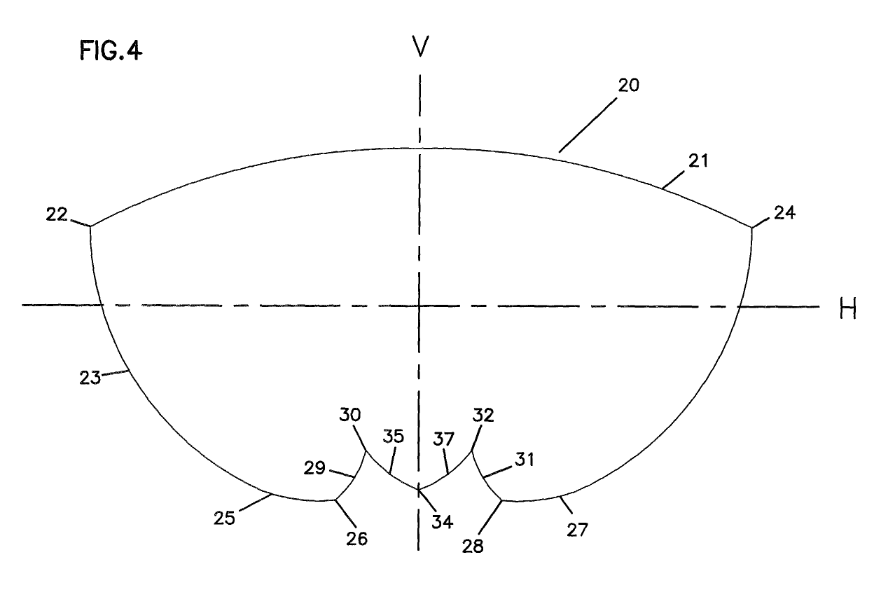

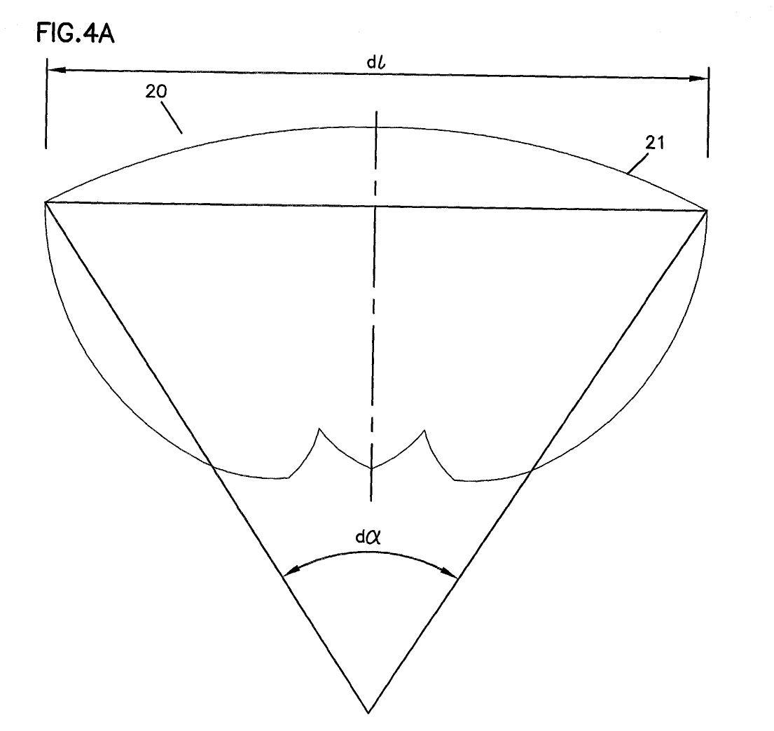

Cardiac support devices of the invention are formed from a two-dimensional starting material, pattern 20. As seen in FIG. 4, pattern 20 comprises a proximal edge 21, first and second proximal regions 22 and 24, and a seam edge 23. Seam edge 23 comprises first and second sides 25 and 27, first and second upward inflection regions 26 and 28, first and second upward edges 29 and 31, first and second downward inflection regions 30 and 32, first and second downward edges 35 and 37, and intersection point 34.

In one embodiment, pattern 20 is symmetrical along a vertical axis V-V. Horizontal axis H-H is transverse to vertical axis V-V. In one embodiment, horizontal axis H-H intersects vertical axis V-V such that the length L₁ of vertical axis V-V above horizontal axis H-H is substantially the same as (i.e. within 5-10%) the length L₂ of vertical axis V-V below horizontal axis H-H. It should be noted that pattern 20 is generally not symmetrical along horizontal line H.

Pattern 20 includes a proximal edge 21, and a seam edge 23. Proximal edge 21 will form the upper rim 13 of jacket 10 once constructed. Proximal edge 21 generally has a concave shape relative to horizontal axis H.

In one embodiment, proximal edge 21 can be further described by its radius of curvature. The radius of curvature of proximal edge 21 can be defined, for example, by dividing the change in the angle (dα) by the change in the length (dL) as illustrated in FIG. 4a. Although proximal edge 21 need not have a constant radius of curvature over its entire length, an estimate of the radius of curvature, measured as explained above for example, can be used in describing it.

The radius of curvature of proximal edge 21 is dependent at least in part on the final size of the resulting jacket 10. Preferably, the radius of curvature of proximal edge 21 for clinical heart sizes is between about 11 inches and about 29 inches. With jackets 10 designed for larger heart sizes having a larger radius of curvature of proximal edge 21.

Table 1 below represents preferred embodiments of the invention. Table 1 gives values of the radius of curvature of proximal edge 21 for jackets 10 with specific surface areas.

TABLE 1

| | |
|---|---|
| | Radius of Curvature of |
| Surface Area | proximal edge 21 |

| inches² | cm² | inches | cm |
|---|---|---|---|
| 67 | 430 | 14 | 36 |
| 82 | 530 | 12 | 30 |
| 89 | 570 | 12-14 | 30-36 |
| 104 | 670 | 13-15 | 33-38 |
| 115 | 740 | 12-13 | 30-33 |
| 133 | 860 | 16 | 40 |
| 157 | 1010 | 21-29 | 53-74 |
| 173 | 1120 | 23 | 58 |

Referring to FIG. 4, seam edge 23 can be further defined as including a first side 25 and side 27 that are defined by vertical axis V-V. As indicated above, in one embodiment, pattern 20 is symmetrical across vertical axis V-V, such that first side 25 and second side 27 are substantially the same shape. The path of first side 25 and second side 27 thus can be explained in the same manner with respect to proximal edge 21 and verticle line V. First and second lines 25 and 27 begin at the first and second proximal regions 22 and 24. From the first and second proximal regions 22 and 24, first and second sides 25 and 27 extend towards intersection point 34. Intersection point 34 is the point where vertical line V and seam edge 23 intersect. The first side 25 and second side 27 generally curve inward toward vertical line V.

First side 25 and second side 27 can be further described by the radius of curvature. Because pattern 20 is symmetrical across vertical axis V-V, the radius of curvature of first side 25 is substantially the same as second side 27. the radius of curvature of first side 25 and second side 27 can be measured in the same way as that of proximal edge 21. Generally, as you progress down first side 25 and second side 27 away from first and second proximal regions 22 and 24, the radius of curvature of first side 25 and second side 27 decreases.

The radius of curvature of first side 25 and second side 27 is dependent at least in part on the final size of the resulting jacket 10. Preferably, the radius of curvature of first side 25 and second side 27 for clinical heart sizes is between about 2 inches and a straight line. As the curvature of a line decreases and the line becomes straight, the radius of curvature will become increasingly larger, with a straight line having a radius of curvature of infinity. With jackets 10 designed for larger heart sizes having a larger radius of curvature of first side 25 and second side 27.

Table 2 below represents preferred embodiments of the invention. Table 2 gives exemplary values of the radius of curvature for the first and second sides 25 and 27 for jackets 10 with specific surface areas.

TABLE 2

| | |
|---|---|
| | Radius of Curvature of first and |
| Surface Area | second sides 25 and 27 |

| inches² | cm² | inches | cm |
|---|---|---|---|
| 67 | 430 | 3-18 | 8-46 |
| 82 | 530 | 4-16 | 10-40 |
| 89 | 570 | 4-20 | 10-51 |
| 104 | 670 | 3-20 | 8-51 |
| 115 | 740 | 5-52 | 13-132 |
| 133 | 860 | 5-straight line | 13-straight line |
| 157 | 1010 | 3-straight line | 8-straight line |
| 173 | 1120 | 5-15 | 13-132 |

The advancement of first side 25 and second side 27 continues until a region along first and second sides 25 and 27, referred to as first and second upward inflection regions, 26 and 28 respectively. At the first and second upward inflection regions 26 and 28, first and second upward edges 29 and 31 begin. First and second upward edges 39 and 31 generally extend towards proximal edge 21.

First upward edge 29 and second upward edge 31 can also be further described by the radius of curvature. Because pattern 20 is generally symmetrical across vertical axis V-V, the radius of curvature of first upward edge 29 is substantially the same as second upward edge 31. The radius of curvature of first upward edge 29 and second upward edge 31 can be measured in the same way as that of proximal edge 21.

The radius of curvature of first upward edge 29 and second upward edge 31 is dependent at least in part on the final size of the resulting jacket 10. Preferably, the radius of curvature of first upward edge 29 and second upward edge 31 for clinical heart sizes in between about 2 inches and a straight line. As the curvature of a line decreases and the line becomes straight, the radius of curvature will become increasingly larger, with a straight line having a radius of curvature of infinity. With jackets 10 designed for larger heart sizes having a larger radius of curvature of first upward edge 29 and second upward edge 31.

Table 3 below represents preferred embodiments of the invention. Table 3 gives exemplary values of the radius of curvature for the first upward edge 29 and second upward edge 31 for jackets 10 with specific surface areas.

TABLE 3

| | |
|---|---|
| | Radius of Curvature of first |
| | and second upward edges |
| Surface Area | 29 and 31 |

| inches² | cm² | inches | cm |
|---|---|---|---|
| 67 | 430 | 4 | 10 |
| 82 | 530 | 3-4 | 8-10 |
| 89 | 570 | 2 | 5 |
| 104 | 670 | 5 | 13 |
| 115 | 740 | straight line | straight line |
| 133 | 860 | 6 | 15 |
| 157 | 1010 | 7 | 18 |
| 173 | 1120 | 7 | 18 |

The advancement of first upward edge 29 and second upward edge 31 continues until first and second downward inflection regions 30 and 32. At first and second downward inflection regions, the first and second downward edges 35 and 37 begin. First and second downward edges 35 and 37 extend away from proximal edge 21. First and second downward edges 35 and 37 continue advancing away from proximal edge 21 until they intersect at the intersection point 34.

First downward edge 35 and second downward edge 37 can also be further described by the radius of curvature. Because pattern 20 is generally symmetrical across verticle axis V-V, the radius of curvature of first downward edge 35 is substantially the same as second downward edge 37. The radius of curvature of first downward edge 35 and second downward edge 37 can be measured in the same way as that of proximal edge 21.

The radius of curvature of first downward edge 35 and second downward edge 37 is dependant at least in part on the final size of the resulting jacket 10. Preferably, the radius of curvature of first downward edge 35 and second downward edge 37 for clinical heart sizes is between about 2 inches and a straight line. As the curvature of a line decreases and the line becomes straight, the radius of curvature will become increasingly larger, with a straight line having a radius of curvature of infinity. With jackets 10 designed for larger heart sizes having a larger radius of curvature of first downward edge 35 and second downward edge 37.

Table 4 below represents preferred embodiments of the invention. Table 4 gives exemplary values of the radius of curvature for the first downward edge 35 and second downward edge 37 for jackets 10 with specific surface areas.

TABLE 4

| | |
|---|---|
| | Radius of Curvature of |
| | first and second down- |
| Surface Area | ward edges 35 and 37 |

| inches² | cm² | inches | cm |
|---|---|---|---|
| 67 | 430 | 4 | 10 |
| 82 | 530 | straight | straight |
| 89 | 570 | 3 | 8 |
| 104 | 670 | 4 | 10 |
| 115 | 740 | 3 | 8 |
| 133 | 860 | 6 | 15 |
| 157 | 1010 | 4 | 10 |
| 173 | 1120 | 5 | 13 |

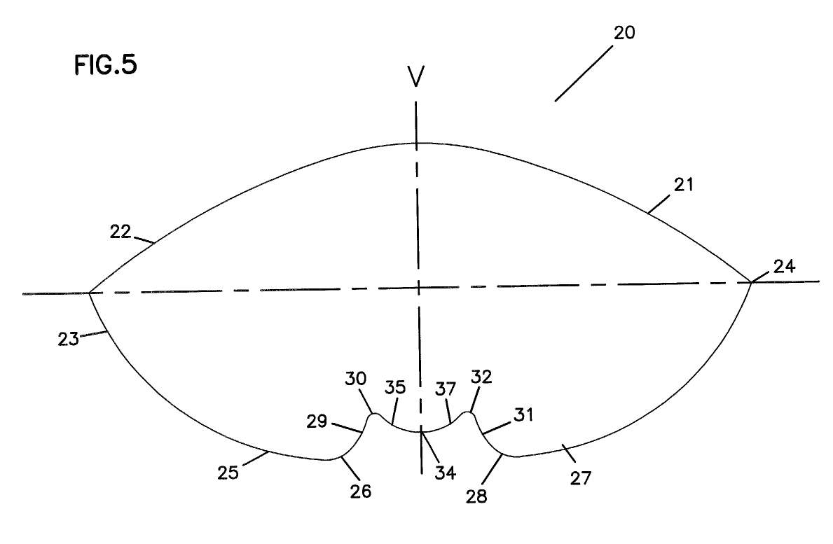

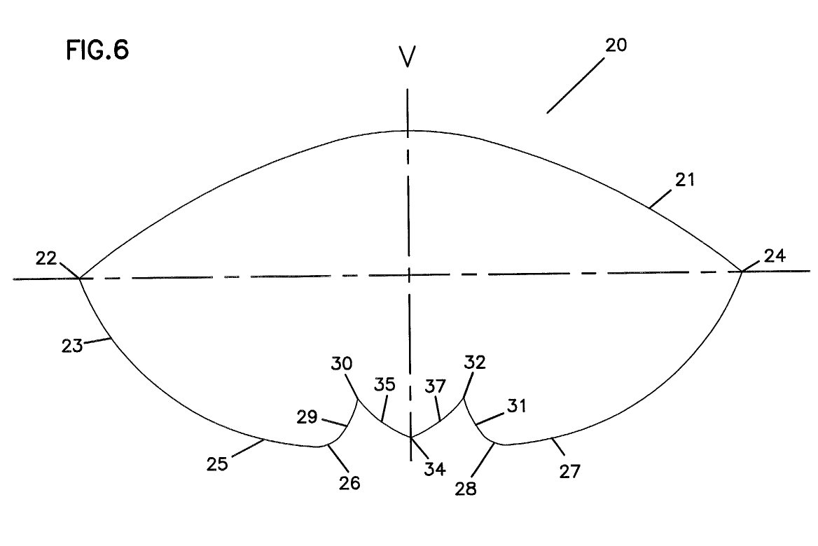

First and second proximal regions 22 and 24, first and second upward inflection regions 26 and 28, and first and second downward inflection regions 30 and 32 can, but need not be able to be defined as a point on seam edge 23. In other words, there can be points along seam edge 23 where seam edge 23 changes direction, as are present in FIG. 5, there can be curves on seam edge 23 that encompass a change in direction for seam edge 23, as illustrated in FIG. 6.

D. Method of Making a Cardiac Support Device

Cardiac support devices, an example of which is jacket 10 are generally constructed from pattern 20 as described below.

Pattern 20 generally comprises a two-dimensional material. Examples of materials can include fabric, knit materials, and intertwined fibers, for example, fibers intertwined as a knit or weave. In a preferred embodiment, the jacket 10 material is a knit material. These materials can be made of polyester, polytetrafluoroethylene (PTFE), expanded PTFE (ePTFE) and polypropylene for example. In one embodiment, a fabric constructed of 70 Denier multifilament polyester yarns is utilized for the material of pattern 20.

Pattern 20 can be formed from the two-dimensional material in a number of ways, including but not limited to cutting, or shearing. Cutting can be accomplished by a scissors, a punch, a thermal blade, or a laser.

The construction of a cardiac support device, such as jacket 10 from pattern 20 can be accomplished by folding said pattern 20 along vertical axis V-V. Once pattern 20 is folded in this manner, seam edge 23 will generally be substantially similar and adjacent to itself. For example, first side 25 will be adjacent second side 27, first upward edge 29 will be adjacent second upward edge 31, and first downward edge 35 will be adjacent second downward edge 37. A seam is then formed that joins these adjacent structures to each other, the seam is adjacent to the entire length of seam edge 23. There is no seam formed along proximal edge 21. In one embodiment of the invention, proximal edge 21 includes a hem. The hem of proximal edge 21 creates a finished edge for attaching sutures to the jacket 10 when implanting it.

The seam that is used to join seam edge 23 to itself can be formed of any thread like material. Examples of types of threads that can be used include but are not limited to surgical suture materials such as polyester (PET) and polytetrafluoroethylene (PTFE). In one embodiment 6-0 polyester suture is used.

The action of forming the seam to join seam edge 23 to itself can be accomplished by any method known to those of skill in the art. For example, it can be sewn, glued, or welded, preferably, it is sewn with a locking stitch such as a zig zag stitch. A locking stitch is preferred because it minimizes the possibility of a cut unraveling the jacket 10.

E. Cardiac Support Devices

Cardiac support devices made using a pattern of the invention and/or a method of the invention have reduced amounts of material distortion. Material distortion, as used herein refers to areas of the jacket 10 where the material is stretched, bunched, or otherwise extended in a fashion that is not desired for the functioning of the jacket 10. The functioning of the jacket 10 can suffer because of these material distortions because they can make the material less compliant.

Cardiac support devices made using a pattern of the invention and/or a method of the invention also have a minimal amount of seam length. Minimizing the number or length of seams in the jacket 10 can be desirable because it minimizes the area where necrosis or inflammation may take place. Necrosis or inflammation can cause areas of increased fibrosis, and/or cause areas of the jacket 10 to adhere to other tissues and or organs.

In one embodiment of the invention, the amount of the seam length can be quantified by the ratio of the seam length to the surface area of the finished jacket. Preferred embodiments of the invention have minimal values for this ratio. Examples of preferred values of the ratio of seam length to surface area for jackets designed for different sized hearts can be seen in Table 5 below.

TABLE 5

| Seam Length (L) | Surface Area (A) | |
|---|---|---|
| inches | inches² | L/A |
| 10.5 | 67.0 | 0.16 |
| 11.3 | 82.3 | 0.14 |
| 14.7 | 88.7 | 0.17 |
| 12.3 | 104.2 | 0.12 |
| 12.2 | 115.0 | 0.11 |
| 14.1 | 133.2 | 0.11 |
| 16.1 | 157.2 | 0.10 |
| 17.2 | 173.4 | 0.10 |

Jackets 10 made using patterns 20 of the invention and/or methods of the invention generally have a seam length (inches) to surface area (inches²) ratio of from about 0.1/inch to about 0.2/inch (about 0.04/cm to about 0.08/cm), preferably a seam length to surface area ratio of from about 0.10/inch to about 0.17/inch (about 0.039/cm to about 0.067/cm), and most preferably a seam length to surface area ratio of from about 0.10/inch to about 0.11/inch (about 0.039/cm to about 0.043/cm).

From the foregoing detailed description, the invention has been described in a preferred embodiment. Modifications and equivalents of the disclosed concepts are intended to be included within the scope of the appended claims.

What is claimed is:

1. A pattern for a three dimensional cardiac support device comprising a proximal edge having a concave shape; and a seam edge comprising:
a. first and second sides, wherein said first and second sides are substantially symmetrical along a vertical axis;
b. first and second upward inflection regions;
c. first and second downward inflection regions; and
d. intersection point.

2. The pattern of claim 1, wherein said proximal edge has a radius of curvature.

3. The pattern of claim 2, wherein said radius of curvature is between about 11 inches and about 29 inches.

4. The pattern of claim 3, wherein said radius of curvature is dependent upon the final size of the three dimensional cardiac support device.

5. The pattern of claim 1, wherein said first and second sides have a radius of curvature.

6. The pattern of claim 5, wherein said radius of curvature is between about 2 inches and a straight line.

7. The pattern of claim 6, wherein said radius of curvature is dependent upon the final size of the three dimensional cardiac support device.

8. The pattern of claim 1, wherein said seam edge further comprises first and second upward edges.

9. The pattern of claim 8, wherein said first and second upward edges are substantially symmetrical along a vertical axis.

10. The pattern of claim 9, wherein said first and second upward edges have a radius of curvature.

11. The pattern of claim 10, wherein said radius of curvature is between about 2 inches and about 8 inches.

12. The pattern of claim 11, wherein said radius of curvature is dependent upon the final size of the three dimensional cardiac support device.

13. The pattern of claim 1, wherein said seam edge further comprises first and second downward edges.

14. The pattern of claim 13, wherein said first and second downward edges are substantially symmetrical along a vertical axis.

15. The pattern of claim 14, wherein said first and second downward edges have a radius of curvature.

16. The pattern of claim 15, wherein said radius of curvature is between about 2 inches and about 6 inches.

17. The pattern of claim 16, wherein said radius of curvature is dependent upon the final size of the three dimensional cardiac support device.

18. A three dimensional cardiac support device comprising:
a. a pattern, wherein said pattern is substantially symmetrical along a vertical axis, and wherein said pattern comprises first and second downward inflection regions;
b. a seam wherein said seam is formed by folding said pattern across said vertical axis and joining said seam edge to itself.

19. The device of claim 18, wherein said seam comprises thread.

20. The device of claim 19, wherein said thread is polyester surgical suture.

21. The device of claim 20, wherein said thread is 6-0 size.

22. The device of claim 18, wherein said seam is formed by sewing, gluing, or welding.

23. The device of claim 22, wherein said seam is formed by sewing.

24. The device of claim 23, wherein said seam is formed by zig-zag stitches.

25. A three dimensional cardiac support device formed from a two-dimensional material, wherein said three-dimensional cardiac support device comprises seams having a seam length, and wherein said three dimensional cardiac support device has a surface area, wherein the ratio of the seam length to the surface area is from about 0.1/inch to about 0.2/inch.

26. The cardiac support device of claim 25, wherein said ratio of seam length to surface area is from about 0.10/inch to about 0.17/inch.

27. The cardiac support device of claim 26, wherein said ratio of seam length to surface area is from about 0.10/inch to about 0.11/inch.

28. A method of making a three-dimensional cardiac support device comprising the steps of:
a. cutting a two-dimensional pattern of material, wherein said pattern comprises
i. first and second sides, wherein said first and second sides are substantially symmetrical along a vertical axis;
ii. first and second upward inflection regions;
iii. first and second downward inflection regions; and
iv. intersection point; and
b. joining said first and second sides to each other to form said three dimensional cardiac support device having a surface area, wherein said joining creates a seam having a length,
wherein said three-dimensional cardiac support device has a ratio of seam length to surface area between about 0.1/inch and about 0.2/inch.
